(19) United States
(12) Patent Application Publication
(10) Pub. No.: US 2009/0178683 A1
(43) Pub. Date: Jul. 16, 2009

(54) DEVICE FOR STABLE SPATIAL FIXATION AND CENTRAL DRILLING WITH DISTAL INTERLOCKING SCREWS IN LIMB PROCEDURES

(76) Inventors: Lior Dayan, Nesher (IL); Roey Shafrir, Modiin (IL); Yves Belleli, Migdal Haemek (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/298,055

(22) PCT Filed: Apr. 25, 2007
(86) PCT No.: PCT/IL07/00509

§ 371 Date: Oct. 22, 2008

Related U.S. Application Data
(60) Provisional application No. 60/784,492, filed on Apr. 24, 2006.

(51) Int. Cl. A61G 15/00; A61B 17/58

(52) U.S. Cl. 128/845; 606/96

(57) ABSTRACT

The device of the present invention provides satisfactory and comfortable three-dimensional spatial holding of an injured limb and allows for the spatial transition of the limb from position to position in accordance with the surgeon's requirements comfortably, easily and within a short time period. Further, the device enables the temporary affixing of fragments of fracture appositionally from before the patient is put into the ambulance, up to the moment the patient reaches the operating theater and even onto the operating table if necessary. The device enables speedy, easy and convenient access to the open wounds in open fractures. It provides a situation allowing a dynamic follow-up of the status of an injured limb up to arriving in the operating theatre and determining decisions on the basis of observation such as for example, but not limited to, swelling, ischemia and development of pressure in the limb's compartment.

### FIELD AND BACKGROUND OF THE INVENTION

The present invention relates to devices for stabilizing an injured limb and, in particular, it concerns a device for the stabilization of the injured limb at the time of initial contact by medical personnel, during transportation of the patient and on the operating table.

Limb operations are among the most common procedures, among which trauma operations are the most frequent. A number of unique problems face the orthopedic surgeon in orthopedic surgery when engaged in limb procedures.

The surgeon needs to hold the limb at various spatial angles so that he can reach any location on which he needs to operate. To do this he requires an additional pair of hands for gripping the foot while conveying instructions to his or her assistant regarding the manner in which the limb should be held spatially.

During the course of the procedure, the transition from one position to another is very frequent. Furthermore, in many of the limb procedures X-ray imaging is performed. For the purpose of producing a satisfactory X-ray image, the photographed region must be fixed spatially so that it will not move; otherwise, the result will be blurred. For this purpose, the surgeon's assistant or the surgeon himself has to hold the limb in various positions while not avoiding exposure to radiation. The instability also causes an unnecessary repeat of photographing and so prolonging the procedure and exposing the team and the patient to unnecessary radiation.

In intramedullary nailing procedures spatial holding problems arise too as described above, as well as a problem of central drilling for distal interlocking screws. Both proximal and distal interlocking screw fixation are accepted practice nowadays. Proximal interlocking is relatively simple since for each nail a special coordinator exists that fits on the proximal part of the nail and “predicts” precisely the location of the proximal holes. However, locating the distal locking holes is far more complex. The reason is that while moving in the marrow cavity the nail twists around its longitudinal axis in addition to bending along its other axes due to the huge pressure and to each bone's unique contour. The result is that it is not possible to predict the exact locations of the target holes for the distal interlocking screws by means of a coordinator, such as exists for proximal locking holes.

This problem is undoubtedly one of the most time consuming stages of the procedure. To date a number of methods are used for centralizing drilling which function according to the “trial and error” method, and in cooperation between the X-ray technician and the surgeon, for example, “a transparent drill” whose tip is made of radiolucent material. This procedure quite often consumes considerable time and numerous X-rays and so forth, including excess exposure of the operating team and the anesthetized patient to radiation.

With regard to long bone fractures, a number of things happen from the time the injury occurs up to the moment the patient is brought into the operating room. Firstly, the patient is moved into the ambulance and jostled to the emergency room. Secondly, there is the examination in the emergency room and bandaging or setting of a temporary splint. Thirdly, the time that passes from the time of diagnosis up to the time of the operation. During this time the bone fragments are in an appositional flail situation, which promotes blood loss and skin necrosis, as well as damage to the soft tissues, release of immune mediators and fat emboli into the circulation, and most important of all, the suffering and pain of the patient.

The orthopedic damage-control approach nowadays is based on the need to first bring the fragments of the fracture to a position of appositional immovability in the Operating Room (OR) setting.

Another problem that arises in open fractures is that various doctors who arrive at different stages are inclined to open the bandaging to observe the injury. In order to do so, they have to actually open the entire fixation (if it concerns the type of splint in use today), which exposes the injury to further infection and additional movement of the fragments.

There is, therefore, a need for a device that is deployable by emergency response personnel for the stabilization of the injured limb at the time of initial contact, used during transportation of the patient to a medical treatment facility and used on the operating table by a surgeon during medical treatment of the injury.

### SUMMARY OF THE INVENTION

The present invention is a device for the stabilization of the injured limb from initial contact by medical personnel, during transportation and onto the operating table

According to the teachings of the present invention there is provided, A device for stabilizing a limb, the device comprising: a) a limb attachment portion configured for attachment to the limb so as to stabilize at least a portion of the limb; and b) a base portion upon which the limb attachment portion is removably attachable so as to provide a mobile deployment of the limb attachment portion wherein the limb attachment portion is removed from the base portion and a static deployment of the limb attachment portion wherein the limb attachment portion is attached to the base portion.

According to a further teaching of the present invention, the limb attachment portion provides rotation about at least one first axis of rotation, and second axis of rotation that is perpendicular to the first axis of rotation, the second axis lying in a plane that is parallel to a plane in which the first axis of rotation lies.

According to a further teaching of the present invention, the limb attachment portion provides rotation about a third axis of rotation that is perpendicular to both the first and the second axes of rotation.

According to a further teaching of the present invention, the third axis of rotation intersects at least one of the first and the second axes of rotation.

According to a further teaching of the present invention, there is also provided attachment of at least one surgical accessory to the limb attachment portion.

According to a further teaching of the present invention, the at least one surgical accessory is a drill guide.

According to a further teaching of the present invention, the limb attachment portion is longitudinally adjustable for use with limbs of differing sizes.

There is also provided according to the teachings of the present invention, a device for stabilizing a limb, the device comprising: a) a limb attachment portion configured for attachment to the limb so as to stabilize at least a portion of the limb; and b) a base portion upon which the limb attachment portion is rotatably attachable such that the limb attachment portion provides rotation about at least one first axis of rotation, a second axis of rotation that is perpendicular to the first axis of rotation, the second axis lying in a plane that is parallel to a plane in which the first axis of rotation lies, and a third axis of rotation that is perpendicular to both the first and the second axes of rotation.

According to a further teaching of the present invention, the third axis of rotation intersects at least one of the first and the second axes of rotation.

There is also provided according to the teachings of the present invention, a method for stabilizing an injured limb of a patient from a first contact by medical personnel until completion of surgical treatment of the injury, the method comprising: a) placement of a limb attachment portion configured or attachment to the limb so as to stabilize at least a portion of the limb; b) transference of the patient to mode of transportation for transportation of the patient to a medical treatment facility; c) transport of the patient to the medical treatment facility; d) transference of the patient to a medical treatment site; and e) attachment of the limb attachment portion to a base portion so as to provide static support of the limb during the surgical treatment of the injury.

According to a further teaching of the present invention, there is also provided providing rotation of the limb about at least one first axis of rotation, a second axis of rotation that is perpendicular to the first axis of rotation, the second axis lying in a plane that is parallel to a plane in which the first axis of rotation lies, and a third axis of rotation that is perpendicular to both the first and the second axes of rotation.

There is also provided according to the teachings of the present invention, a drill guiding assembly comprising at least one of drill guide support rod extending from an attachment base so as to allow rotation of the drill guide support rod about a central axis of the attachment base, and at least one guide element mechanically linked to the at least one drill guide support rod so as to longitudinal displacement of the at least one guide element along a length of the drill guide support rod and rotational displacement of the at least one guide element about a central axis of the drill guide support rod.

According to a further teaching of the present invention, the at least one drill guide support rod is configured as a plurality of drill guide support rods slidingly and rotationally interconnected in a series so as to form a drill guide support frame.

According to a further teaching of the present invention, the attachment base is interconnected to a device for stabilizing a limb.

According to a further teaching of the present invention, the guide element is configured as at least one guide ring.

### BRIEF DESCRIPTION OF THE DRAWINGS

The invention is herein described, by way of example only, with reference to the accompanying drawings, wherein:

FIG. 1 is an isometric view of a preferred embodiment of a device for the stabilization of the injured limb constructed and operative according to the teachings of the present invention;

FIG. 2 is an isometric view of the lower portion of the embodiment of FIG. 1;

FIGS. 3 and 4 are isometric views of the upper portion of the embodiment of FIG. 1; and

FIG. 5 is an isometric view of a drill guiding assembly attached to the embodiment of FIG. 1.

### DESCRIPTION OF THE PREFERRED EMBODIMENTS

The present invention is a device for the stabilization of the injured limb from initial contact by medical personnel, during transportation and onto the operating table

The principles and operation of a device for the stabilization of the injured limb according to the present invention may be better understood with reference to the drawings and the accompanying description.

By way of introduction, the device of the present invention provides satisfactory and comfortable three-dimensional spatial holding of an injured limb and allows for the spatial transition of the limb from position to position in accordance with the surgeon's requirements comfortably, easily and within a short time period. Since it is know that there is a direct correlation between the length of the medical procedure and healing time, by shortening the time of the of the operation, the device of the present invention also aids in the healing process.

Further, the device enables the temporary affixing of fragments of fracture appositionally from before the patient is put into the ambulance, up to the moment the patient reaches the operating theater and even onto the operating table if necessary Attachment of the device of the present invention to an injured limb helps to reduce blood loss and skin necrosis. Therefore, this ability to deploy the device upon first contact with the patient and maintain sustained attachment during transportation of the patient to the medical facility, through pre-operational procedures and the operation itself addresses the issues of blood loss and skin necrosis in way not currently available.

The device enables speedy, easy and convenient access to the open wounds in open fractures. It provides a situation allowing a dynamic follow-up of the status of an injured limb up to arriving in the operating theatre and determining decisions on the basis of observation such as for example, but not limited to, swelling, ischemia and development of pressure in the limb's compartment.

The device will provide assistance in a number of procedures, such as but not limited to, urgent trauma procedures, and elective procedures on upper and lower limbs. Therefore, the terms “injured limb” and “limb” may be used interchangeably herein since the device of the present invention may be used with equal benefit during medical procedures involving either an injured limb or a non-injured limb.

The device of the present invention enables centralizing drilling of distal interlocking screws in intramedullary nailing procedures, so that the entire process is undertaken in a much simpler and easier manner within a minimal time span. Aligning the drilling path of distal interlocking screws is accomplished in a manner that does not expose the surgeon holding the limb to radiation, and that will provide stability and save from having to redo unsuccessful X-rays. X-rays may be taken without the need for someone to hold the limb, as is in practice today. Therefore, procedure time is reduced considerably. This significantly shortens the procedure time and exposure of the patient, the doctor and the entire medical team to X-ray radiation.

The devise of the present invention also provides some functional advantages. Specifically, the device of the present invention is light and convenient for transportation. The device can be easily dismantled and reassembled, and therefore is useful in a variety of locations, such as but not limited to, ambulances, stretchers, airplanes, helicopters and any number of emergency medical situations.

Additionally, the device of the present invention may be partially metallic and partially made of radiolucent substances preventing interference to X-rays. It can be dismantled, washed and autoclaved for repeated use. further, the design of the device does not interfere with arterial tourniquets, which are often used in limb procedures.

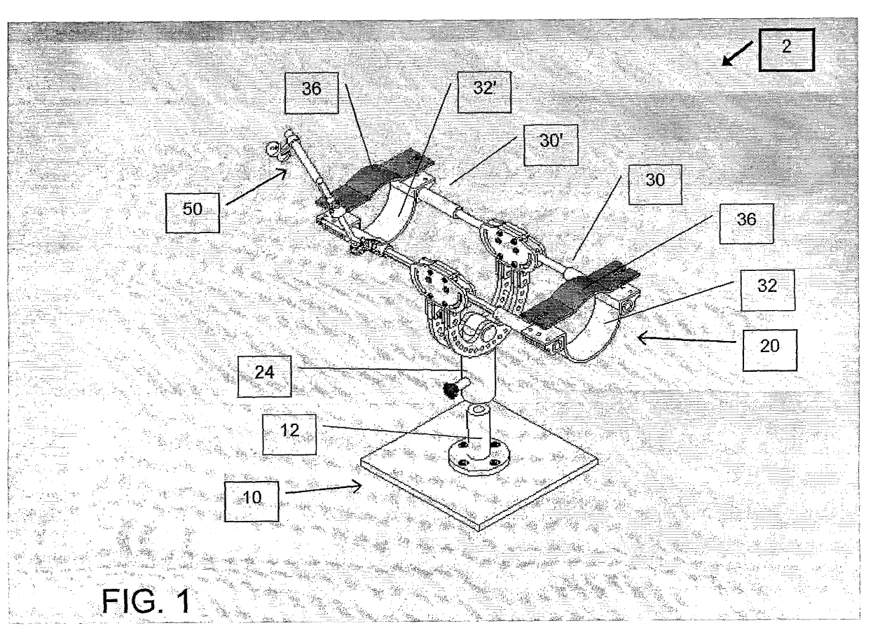

Referring now to the drawings, FIG. 1 illustrates a preferred embodiment of the device of the present invention, generally referred with the reference numeral 2, that includes a lower portion having a base 10, a limb attachment portion 20 configured to hole the injured limb, and when needed a drill guiding assembly 50 may be attached to the upper portion.

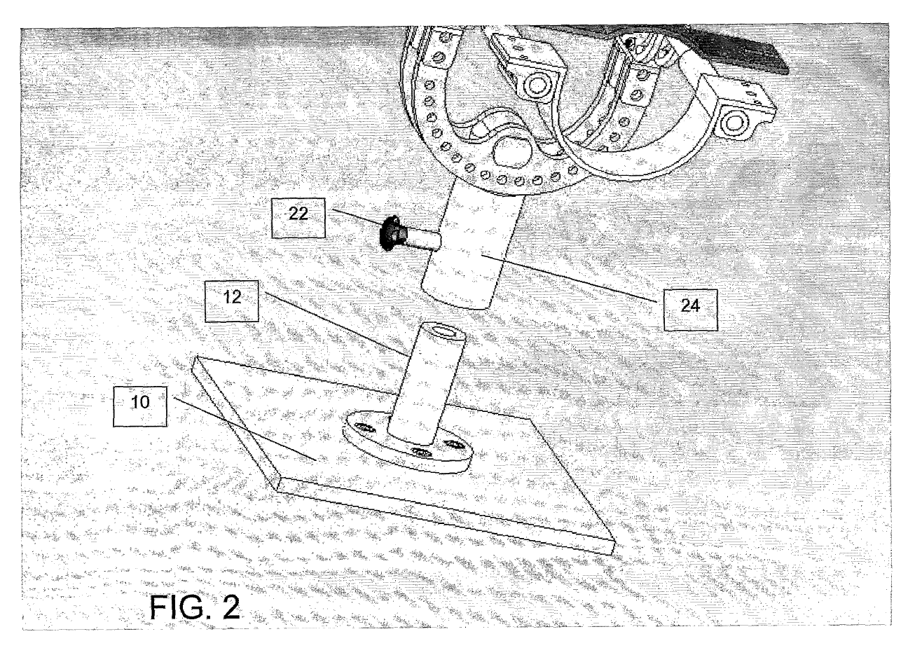

As illustrated in FIG. 2, the base 10 includes a post 12 upon which the hollow extension 24 is mounted so as to attach the limb attachment portion 20 to the base 10. This attachment arrangement provides both height adjustment and 360° rotational adjustment. Once the desired position is achieved, the limb attachment portion 20 may be locked in place using the locking screw 22.

This base arrangement allows the devise to be mobile as in, but not limited to, cases when traveling in an ambulance or while awaiting a procedure. Alternatively, the device may be static by affixing it to, by non-limiting example, a bed, gurney or special ambulance fixture. It will be appreciated that the limb attachment portion 20 may be transferred between any number of bases 10. Therefore, the limb attachment portion 20 may be attached to a patient during the initial contact by medical personnel. Once in an ambulance, the limb attachment portion 20 may be attached to a base 10 mounted in the ambulance for static support of the limb during transport to the medical facility. Upon arrival, the limb attachment portion 20 may be removed for the ambulance base 10 for transfer to the Emergency Room or the Operating Room at which time the limb attachment portion 20 may again be attached to another base 10.

After affixing the limb attachment portion 20 in a static deployment on a base 10, the height of the limb can be controlled (which cannot be done with current splints) so as to adjust the limb's height accordingly above the height of the heart in order to increase venous return and thus reduce edema in the fractured area.

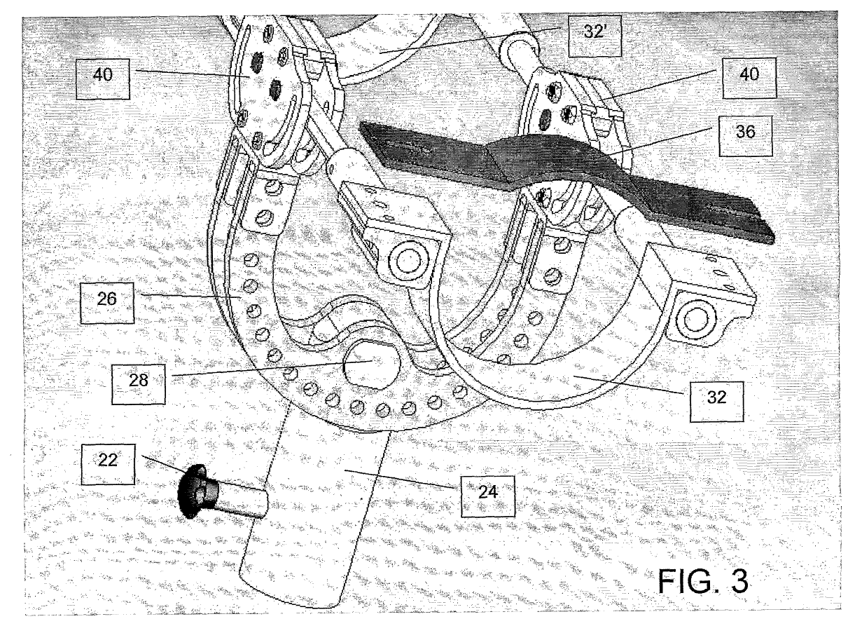

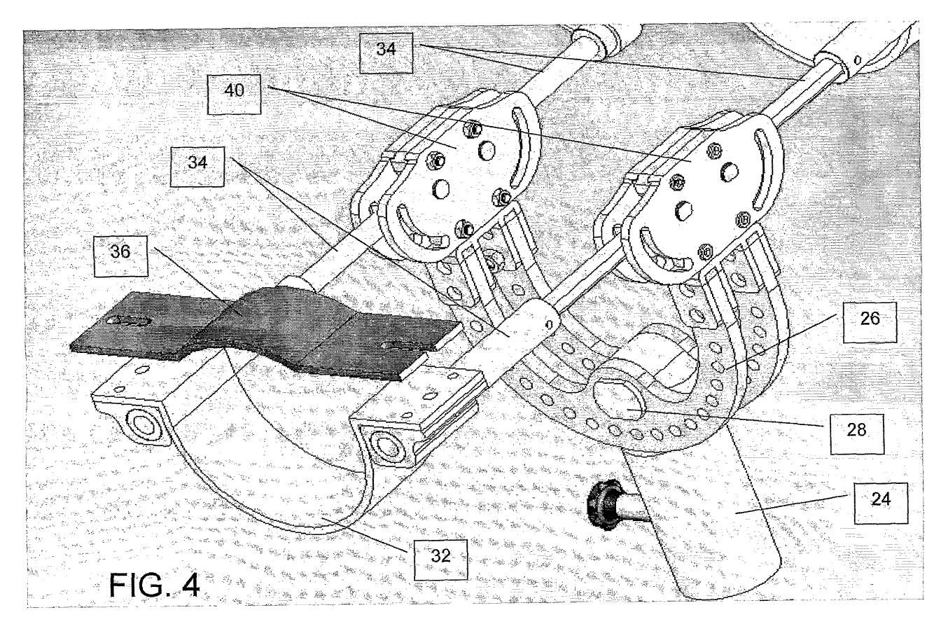

FIGS. 3 and 4 provide a more detailed illustration of the limb attachment portion 20. Rotatably attached to the hollow extension 24 on the axis created by pin 28 is a substantially semi-circular support element 26 that in turn is rotatably attached to the attachment arms 30 and 30′. In this configuration, the device of the present invention provides rotation about at least one of the axes of rotation of the attachment arms 30 and 30′, the axis of rotation of the semi-circular support element 26 about pin 28, and rotation of the entire limb attachment portion 20 about post 12 on the base 10. Therefore, the axis of rotation of the semi-circular support element 26 about pin 28, is perpendicular to the axes of rotation of the attachment arms 30 and 30′, and lies in a plane that is parallel to a plane in which the axes of rotation of the attachment arms 30 and 30′ lie. The axis of rotation of the entire limb attachment portion 20 about post 12 on the base 10 is perpendicular to both axes of rotation of the attachment arms 30 and 30′ and the axis of rotation of the semi-circular support element 26 about pin 28. It will be appreciated that the axis of rotation of the entire limb attachment portion 20 about post 12 on the base 10 intersects the axis of rotation of the semi-circular support element 26 about pin 28.

As illustrated here, preferably, both attachment arms 30 and 30′ are rotatably attached to support element 26 via hinge elements 40. However, during deployment on a patient, only one attachment arm may need to be rotated in order to fit the limb attachment portion 20 in place. The rotation may be incremental across the range of rotation, such as but not limited to, steps of 20°. However, it will be appreciated that rotational attachment of only one of the attachment arms 30 and 30′ is within the scope of the present invention.

Once the limb in place in the limb attachment cuffs 32 and 32′, cuff fasteners 36 are attached to secure the limb in place in the limb attachment cuffs 32 and 32′. It should be noted that in order to prevent pressure sores during prolonged attachment of the attachment portion, it is preferable that the limb attachment cuffs include cushioning material at points of contact. This cushioning material may be permanently affixed or removable. The limb attachment cuffs 32 and 32′, are preferably slidingly attached to the support rods 34 of the attachment arms 30 and 30′. This allows for stretching the limb on either side of the injury if it is necessary or desirable to do so. This also provides an adjustment to allow use on substantially any patient regardless of limb size. That is, the limb attachment portion 20 may be longitudinally adjusted to allow use on limbs of differing sizes. Alternatively, the support rods 34 may be configured to telescope, and thereby allow for longitudinal displacement of the limb attachment cuffs 32 and 32′.

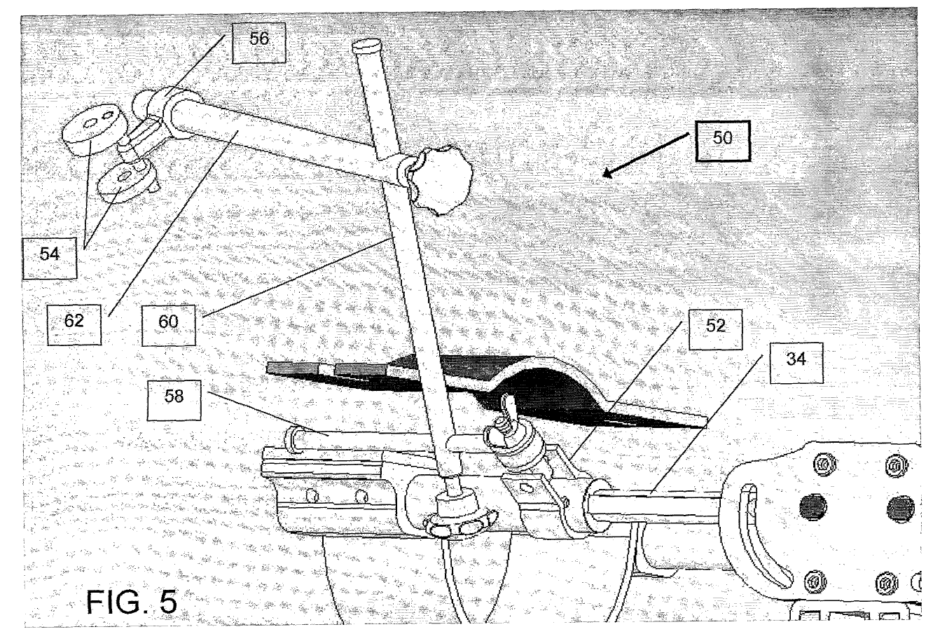

FIG. 5 provides a more detailed illustration of the deployment of a drill guiding assembly 50 to one of the support rods 34, as a non-limiting example of a surgical accessory mounted on the limb attachment portion 20 of the device of the present invention. Drill guiding assembly 50 includes a base clamp element 52 that is deployable on any one of the support rods 34 in a manner that allows for 360° rotation of the base clamp element 52 about the support rod 34, and the base clamp element 52 may be mounted substantially anywhere along the length of the support rod 34. Extending from the base clamp is at least one, and preferably a series, of drill guide support rods 58, 60 and 62. As illustrated here in FIG. 5, each of the three drill guide support rods 58, 60 and 62 are slidingly and rotatably mounted on a previous rod in the series so as to form a drill guide support frame. Additionally, the guide ring clamp 56 on which the guide rings 52 are mounted is also slidingly and rotatably mounted one of the drill guide support rod 62. That is to say, drill guide support rod 58 is attached to the base clamp element 52 in a manner that allows 360° of rotation about the central axis of the base clamp element 52. Drill guide support rod 60 is mounted on drill guide support rod 58 in a manner that allows 360° rotation of drill guide support rod 60 about the central axis of drill guide support rod 58 along substantially the full length of drill guide support rod 58. Likewise, drill guide support rod 62 is mounted on drill guide support rod 60 in a manner that allows 360° rotation of drill guide support rod 62 about the central axis of drill guide support rod 60 along substantially the full length of drill guide support rod 60. Finally, guide ring clamp 56, to which guide rings 54 are attached, is mounted on drill guide support rod 62 in a manner that allows 360° rotation of guide ring clamp 56 about the central axis of drill guide support rod 62 along substantially the full length of drill guide support rod 62. This support frame arrangement provides substantially unlimited positional displacement of the guide rings 54 in relation to the site of the medical procedure. It will be understood that the guide rings may be interchangeable to accommodate the different diameters of standard drilling devices as are known in the art. Further, although the guide element illustrated herein is in the form of rings, this is not intended as a limitation of the present invention, rather as a non-limiting example.

It will be appreciated that the drill guiding assembly 50 is illustrated here in FIG. 5 only as a non-limiting example of a drill guiding assembly according to the teachings of the present invention. The scope of the present invention includes a drill guiding assembly having at least one, and preferably a plurality of, drill guide support rods extending from an attachment base and with at least one guide ring mechanically linked to one of the drill guide support rods.

A non-limiting example of the use of the drill guiding assembly 50 is the drill of the holes for the placement of distal interlocking screws in intramedullary nailing procedures. As in every procedure involving the insertion of remote interlocking screws, a technician marks the target location for the distal end of the holes. Following this, the surgeon adjusts, by trial and error, the two guide rings 54 so that they are precisely aligned to the central axis of the nail hole. The advantage is that the surgeon can perform modifications at any spatial point without having to hold the limb or be exposed to X-ray radiation while checking the alignment. Furthermore, from the moment the target location is established it is fixed in place by the device's hold on the limb. The moment the surgeon observes in the X-that the two rings are correctly positioned, he can drill “blindly” with a standard drill using the rings as a guide.

Alignment for drilling additional holes also becomes far easier. Since the axis of the drilling path is now established, there is only a need to move the rings to align with the target location of the next pair of holes, in a fixed and stable manner. This alignment may be accomplished by any one or combination of adjustments of the support rods 58, 60 and 62, rotation about the base clamp, or displacement of the guide ring clamp 56.

It is worth noting that this device can also be used for, but is not limited to, drilling proximal interlocking screws or for locating the appropriate point for inserting Steinman or Schantz rods in external affixing procedures or for locating drilling holes of plates in mini-invasive procedures in which the plate is inserted in a “blind” manner subcutaneously.

It should also be noted that limb specific accessories for support in more specific medical procedures are within the scope of the present invention. Such limb specific accessories may include, but are not limited to, a foot support configured to support the foot during foot or ankle procedures.

It will be appreciated that the above descriptions are intended only to serve as examples and that many other embodiments are possible within the spirit and the scope of the present invention.

What is claimed is:

1. A device for stabilizing a limb, the device comprising:
(a) a limb attachment portion configured for attachment to the limb so as to stabilize at least a portion of the limb; and
(b) a base portion upon which said limb attachment portion is removably attachable so as to provide a mobile deployment of said limb attachment portion wherein said limb attachment portion is removed from said base portion and a static deployment of said limb attachment portion wherein said limb attachment portion is attached to said base portion.

2. The device of claim 1, wherein said limb attachment portion provides rotation about at least one first axis of rotation, and second axis of rotation that is perpendicular to said first axis of rotation, said second axis lying in a plane that is parallel to a plane in which said first axis of rotation lies.

3. The device of claim 2, wherein said limb attachment portion provides rotation about a third axis of rotation that is perpendicular to both said first and said second axes of rotation.

4. The device of claim 3, wherein said third axis of rotation intersects at least one of said first and said second axes of rotation.

5. The device of claim 1, further including attachment of at least one surgical accessory to said limb attachment portion.

6. The device of claim 5, wherein said at least one surgical accessory is a drill guide.

7. The device of claim 1, wherein said limb attachment portion is longitudinally adjustable for use with limbs of differing sizes.

8. A device for stabilizing a limb, the device comprising:
(a) a limb attachment portion configured for attachment to the limb so as to stabilize at least a portion of the limb; and
(b) a base portion upon which said limb attachment portion is rotatably attachable such that said limb attachment portion provides rotation about at least one first axis of rotation, a second axis of rotation that is perpendicular to said first axis of rotation, said second axis lying in a plane that is parallel to a plane in which said first axis of rotation lies, and a third axis of rotation that is perpendicular to both said first and said second axes of rotation.

9. The device of claim 8, wherein said third axis of rotation intersects at least one of said first and said second axes of rotation.

10. A method for stabilizing an injured limb of a patient from a first contact by medical personnel until completion of surgical treatment of the injury, the method comprising:
(a) placement of a limb attachment portion configured for attachment to the limb so as to stabilize at least a portion of the limb;
(b) transference of the patient to mode of transportation for transportation of the patient to a medical treatment facility;
(c) transport of the patient to said medical treatment facility;
(d) transference of the patient to a medical treatment site; and
(e) attachment of said limb attachment portion to a base portion so as to provide static support of the limb during the surgical treatment of the injury.

11. The method of claim 10, further including providing rotation of the limb about at least one first axis of rotation, a second axis of rotation that is perpendicular to said first axis of rotation, said second axis lying in a plane that is parallel to a plane in which said first axis of rotation lies, and a third axis of rotation that is perpendicular to both said first and said second axes of rotation.

12. A drill guiding assembly comprising at least one of drill guide support rod extending from an attachment base so as to allow rotation of said drill guide support rod about a central axis of said attachment base, and at least one guide element mechanically linked to said at least one drill guide support rod so as to longitudinal displacement of said at least one guide element along a length of said drill guide support rod and rotational displacement of said at least one guide element about a central axis of said drill guide support rod.

13. The drill guiding assembly of claim 12, wherein said at least one drill guide support rod is configured as a plurality of drill guide support rods slidingly and rotationally interconnected in a series so as to form a drill guide support frame.

14. The drill guiding assembly of claim 12, wherein said attachment base is interconnected to a device for stabilizing a limb.

15. The drill guiding assembly of claim 12, wherein said guide element is configured as at least one guide ring.
